(19) United States
(12) Patent Application Publication
(10) Pub. No.: US 2007/0093458 A1
(43) Pub. Date: Apr. 26, 2007

(54) Preparation of paricalcitol and crystalline forms thereof

(76) Inventors: Anchel Schwartz, Rehovot (IL); Alexei Ploutno, Bat-Yam (IL); Koby Wolfman, Petah Tikva (IL); Shlomit Wizel, Petah Tikva (IL)

Correspondence Address: KENYON & KENYON LLP, ONE BROADWAY, NEW YORK, NY, 10004, US

(21) Appl. No.: 11/520,471

(22) Filed: Sep. 12, 2006

Related U.S. Application Data
(63) Continuation-in-part of application No. 11/489,148, filed on Jul. 18, 2006.
(60) Provisional application No. 60/700,477, filed on Jul. 18, 2005.
(60) Provisional application No. 60/716,801, filed on Sep. 12, 2005.

(51) Int. Cl. A61K 31/59; C07C 401/00

(52) U.S. Cl. 514/167; 552/653

(57) ABSTRACT

The present invention is directed to a novel process for preparing Paricalcitol wherein Paricalcitol, dissolved in a solvent, is precipitated from a concentrated or seeded solution. The present invention also provides crystalline forms of paricalcitol and processes for their preparations.

### CROSS REFERENCE TO RELATED APPLICATIONS

The present application is a continuation-in-part application of U.S. patent application Ser. No. 11/489,148, filed Jul. 18, 2006, which claims the benefit of the following U.S. Provisional Patent Application No.: 60/700,477 filed 18 Jul. 2005. The present application also claims the benefit of the following U.S. Provisional Application No. 60/716,801, filed Sep. 12, 2005. The contents of these applications are incorporated herein by reference.

### FIELD OF INVENTION

The present invention is directed to a process for preparing Paricalcitol and to solid state chemistry of Paricalcitol.

### BACKGROUND OF THE INVENTION

Vitamin D is a fat-soluble vitamin. It is found in food, but also can be formed in the body after exposure to ultraviolet rays. Vitamin D is known to exist in several chemical forms, each with a different activity. Some forms are relatively inactive in the body, and have limited ability to function as a vitamin. The liver and kidney help convert vitamin D to its active hormone form. The major biologic function of vitamin D is to maintain normal blood levels of calcium and phosphorus. Vitamin D aids in the absorption of calcium, helping to form and maintain healthy bones.

The 19-nor vitamin D analogue, Paricalcitol (I) (CAS Registry Number 131918-61-1), is characterized by the following formula: 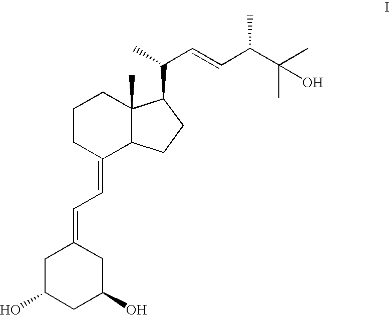
Paricalcitol is a member of the vitamin D family and has found use in the treatment of, for example, secondary hyperparathyroidism, Abrow, A. J. and Coyne, D. W., Treat. Endrocrinol., 1(5) 313-27 (2002) and plaque psoriasis, Br. J. Dermatol., 151(1) 190 (2004).

In the synthesis of vitamin D analogues, a few approaches to obtain a desired active compound have been outlined previously. One of the methods is the Wittig-Homer attachment of a 19-nor A-ring phosphine oxide to a key intermediate bicyclic-ketone of the Windaus-Grundmann type, to obtain the desired Paricalcitol, as is shown for example in U.S. Pat. Nos. 5,281,731 and 5,086,191 of DeLuca.

The synthesis of Paricalcitol requires many synthetic steps which produce undesired by-products. Therefore, the final product may be contaminated not only with a by-product derived from the last synthetic step of the process but also with compounds that were formed in previous steps. In the United States, the Food and Drug Administration guidelines recommend that the amounts of some impurities be limited to less than 0.1 percent.

U.S. Pat. Nos. 5,281,731 and 5,086,191 of DeLuca disclose a purification process of Paricalcitol by using a HPLC preparative method.

As the unwanted products have almost the same structure as the final product, it may be difficult to get a sufficiently pure drug substance, vitamin D analogue, using this route to purify the drug substance. Moreover, the high polarity of Paricalcitol makes it very difficult to purify by HPLC and to recover the solid product. Furthermore, HPLC preparative methods are generally not applicable for use on industrial scale. There remains a need in the art to provide a method of preparing the vitamin D analogue Paricalcitol in a sufficiently pure form which is applicable for use on an industrial scale.

The present invention relates to the solid state crystal structural and physical properties of paricalcitol. Solid state physical properties of a compound are known to be influenced by the solid state crystalline form (crystal structure) of the compound. Solid state physical properties influenced by solid state crystal structure include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important property in the solid state of a compound like paricalcitol that has pharmacological activity that can be influenced by its solid state crystalline structure is the rate of dissolution of the compound in aqueous media, for example gastric fluid. The rate of dissolution of an active pharmaceutical ingredient in a patient's stomach fluid can have therapeutic consequences because, if it is too low, it can be the rate-determining step controlling the rate at which an orally-administered active ingredient reaches the patient's bloodstream. The rate of dissolution is also a consideration in formulating syrups, elixirs and other liquid medicaments. Also, different crystalline forms (polymorphs) can behave differently when compacted and can have different storage stabilities, “shelf lives”.

These practical physical characteristics are influenced by the conformation and orientation of molecules in the unit cell, which defines a particular crystalline (polymorphic) form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric analysis (DSC), and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

U.S. Pat. No. 5,237,110 apparently describes a method for the preparation of paricalcitol from either vitamin D₂ or 25-hydroxy vitamin D₂. In this patent, the final process steps in preparing paricalcitol is as follows: “The residue was dissolved in a 1:1 mixture of 2-propanol and hexane and passed through a Sep Pak column and washed with the same solvent. The solvents were evaporated and the residue purified by HPLC (Zorbax Sil, 6.4×25 cm, 10% 2-propanol in hexane).” Otherwise, U.S. Pat. No. 5,237,110 does not disclose polymorphic forms of paricalcitol, or that paricalcitol could even exist in different polymorphic forms.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

### SUMMARY OF THE INVENTION

In one aspect, the present invention provides a method for purifying Paricalcitol comprising the steps of
a) dissolving Paricalcitol in a solvent;
b) cooling the solution to form a precipitate; and
c) recovering the precipitate.
   Preferably the solvent is selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof, more preferably the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof. Most preferably, the solvent is acetone.

In another aspect, the present invention provides a crystalline paricalcitol form II, having a powder X-ray diffraction pattern comprising peak reflections at about 4.7°, 8.0°, 11.6°, 17.4°, 18.3°, and 19.0°±0.2° 2θ.

In another aspect, the present invention provides a mixture of crystalline paricalcitol forms I and II, having a powder X-ray diffraction pattern comprises peak reflections at about 5.4°, 8.0°, 11.6°, 14.2°, 15.2°, 17.8°, 18.3°, and 19.0°±0.2° 2θ.

In yet another aspect, the present invention provides a process of preparing a mixture of crystalline paricalcitol forms I and II, comprising the steps of
a) dissolving paricalcitol in tert-butanol forming a solution;
b) concentrating the solution;
c) cooling the solution; and
d) recovering the mixture of crystalline paricalcitol forms I and II from the slurry.

In another aspect, the present invention provides a pharmaceutical composition comprising crystalline paricalcitol form II and at least one pharmaceutical acceptable excipient.

In yet another aspect the present invention provides a method of preparing a pharmaceutical composition comprising crystalline paricalcitol form II comprising the steps of
a) providing crystalline paricalcitol comprising crystalline paricalcitol form II; and
b) mixing the crystalline paricalcitol with at least one pharmaceutical acceptable excipient.

The present invention also provides a method of treating a patient suffering from an illness comprising administering to the patient in need thereof a therapeutically effective amount of a pharmaceutical composition comprising crystalline paricalcitol form II and at least one pharmaceutical acceptable excipient.

### BRIEF DESCRIPTION OF THE FIGURES

FIG. 1: XRD Diffractogram of Paricalcitol form I.

FIG. 2: XRD Diffractogram of a mixture of Paricalcitol form II and form I.

FIG. 3: DSC thermogram of Paricalcitol form I.

FIG. 4: DSC thermogram of a mixture of Paricalcitol form II and form I.

FIG. 5: TGA thermogram of Paricalcitol form I.

FIG. 6: TGA thermogram of a mixture of Paricalcitol form II and form I.

### DETAILED DESCRIPTION OF THE INVENTION

In one aspect, the present invention provides a process for purifying Paricalcitol. This process may be practiced without the need for an HPLC preparative method. The process of the invention may be easily applied to an industrial scale. An industrial scale process is that which prepares a batch of at least 5 g of the API, more preferably at least 10 g of the API.

During the preparation of Paricalcitol, various unwanted by-products may be formed, depending on the method employed for its preparation. One of the most common by-products is its C-24 isomer. Another common by-product is its C-14 epimer.

In one aspect, the present invention provides a method for purifying Paricalcitol comprising the steps of
a) dissolving Paricalcitol in a solvent;
b) cooling the solution to form a precipitate; and
c) recovering the precipitate.

The solvent for use in the method of the present invention is preferably selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof, more preferably the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof. Most preferably, the solvent is acetone.

Preferably, the ratio between Paricalcitol and the solvent is about 1:150-1:450 g of Paricalcitol/ml of solvent, more preferably about 1:150-1:250 g of Paricalcitol/ml of solvent, most preferably about 1:150-1:200 g Paricalcitol/ml of solvent. In addition, the step of dissolving Paricalcitol in a solvent is preferably carried out at a temperature of about 25° C. to about 40° C., more preferably at a temperature of about 28° C. to about 34° C.

The solution is preferably filtered after the step of dissolving Paricalcitol in a solvent in the method of the present invention, to obtain a clear solution. The filtration removes solids that have not dissolved in the solvent.

Preferably, the solution is cooled to a temperature of about −45° C. to about −10°C., more preferably about −20° C. to about −15° C., most preferably to a temperature of about −18° C. However, some solvents suitable for use in the method of the present invention freeze at such low temperatures, for example (clean) tert-butanol freezes at temperatures between 24° C. and 26° C. In such cases, the solution is cooled to a temperature above the freezing point so as to maintain the solution in liquid form. Therefore, when tert-butanol is used as a solvent in the method of the present invention, the solution is cooled to a temperature of about 25° C.-27° C.

In one embodiment of the present invention the solution is cooled at a controlled slow rate. Preferably, the solution is cooled at a rate of not more than about 8° C. per hour, more preferably not more than about 4° C. per hour. The cooling of the solution at a slow rate results in decreased amounts, less than about 5000 ppm, of residual solvent in the purified composition. Preferably, cooling the solution at a slow rate reduces the amount of residual solvent to about 800-1500 ppm.

The solution is cooled for a sufficient amount of time to obtain a desirable amount of solids. Preferably, the solution is cooled for a period of about 15 to about 24 hours, more preferably for a period of about 16 to about 20 hours. When tert-butanol is used as the solvent in the method of the present invention, the solution is cooled at a temperature of about 25° C.-27° C. for a period of about 1 to about 4 hours.

In the present invention dissolution of Paricalcitol in a solvent is preferably carried out in a sonicator. The use of sonication while dissolving Paricalcitol enables the use of relatively low amounts of solvent.

In another aspect of the present invention the method further comprises concentrating the solution of Paricalcitol in solvent from step a) before cooling the solution. Preferably, the solution is concentrated to obtain a ratio of about 1:100-1:120 g Paricalcitol/ml of solvent. Therefore, the solution is concentrated in the method of the present invention to reduce its volume to about 0.5 to about 0.9, preferably about 0.6 to about 0.8, times its original volume. Concentrating the solution in the method of the present invention may be carried out using methods know to those skilled in the art. Such methods of concentrating the solution include for example concentration by evaporation, filtration, and dialysis. When the solvent for dissolving Paricalcitol is a mixture of solvents as described above, concentrating the solution of dissolved Paricalcitol in the solvent mixture is optional.

In another aspect of the method of the present invention the method further comprises seeding the solution with crystals either before or during the step of cooling the solution. The solution may be seeded to promote crystallization. Crystals of Paricalcitol may be used as seeds. In one embodiment, both a seeding and a concentrating step is carried out.

The precipitated product may be recovered by conventional means. Preferably, the recovery step includes filtering the cooled solution, and drying it under reduced pressure, preferably in vacuum (pressure of less than 100 mmHg).

The method of the present invention preferably yields about 50% to about 80% of Paricalcitol. Preferably, the Paricalcitol prepared according to the method of the present invention has a purity of at least about 98%, preferably a purity of at least about 98.5% and more preferably a purity of at least about 99%, in weight percent.

Further, it has been discovered that paricalcitol can exist in at least two crystalline forms that differ from each other in the way the molecules of paricalcitol are arranged (packed) in the crystal lattice. The present inventors denominate the two crystalline forms as form I and form II. The solid state crystalline form of paricalcitol can be influenced by controlling the conditions under which the paricalcitol is obtained in solid form.

In another aspect, the present invention provides a crystalline form of paricalcitol denominated form II, characterized by powder X-ray reflections at about 4.7°, 8.0°, 11.6°, 17.4°, 18.3°, and 19.0°±0.2° 2θ. Further, crystalline paricalcitol form II, can be characterized having additional peak powder X-ray reflections at about 6.9°, 9.5°, 12.2°, 12.6°, 13.8°, 20.7°±0.2° 2θ. The present inventors do not observe reflections at these angles in the powder X-ray diffraction diagram (PXRD) of paricalcitol form I.

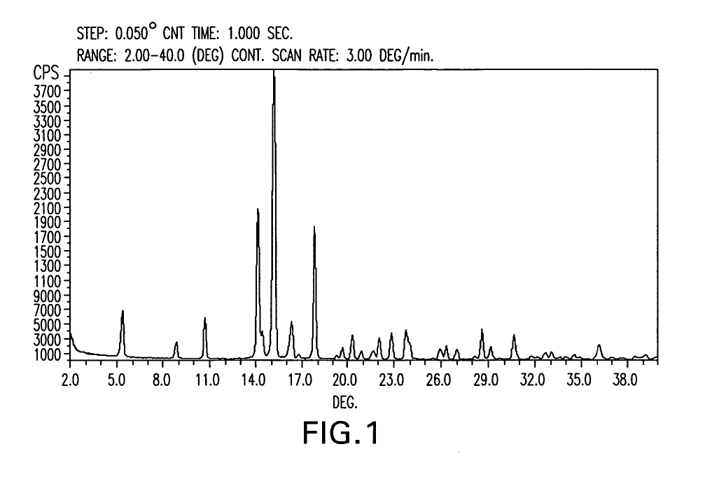

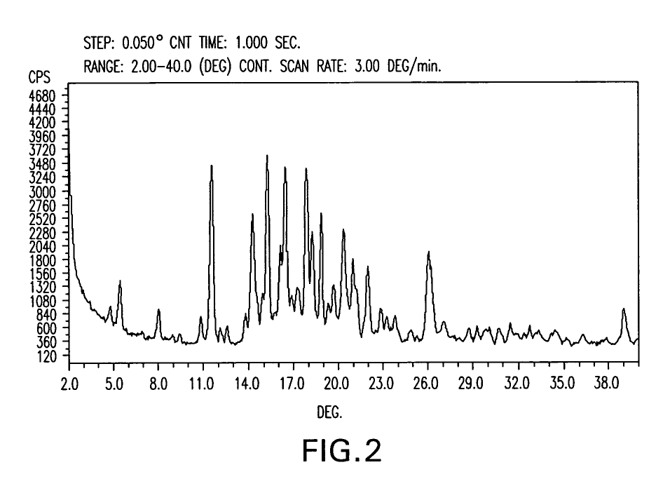

Crystalline paricalcitol form I exhibits powder X-ray reflections at about 5.4°, 10.8°, 14.2°, 15.2°, and 17.8°±0.2° 2θ. FIG. 1 is a representative PXRD of paricalcitol form I. FIG. 2 is a representative PXRD of paricalcitol form II in admixture with form I.

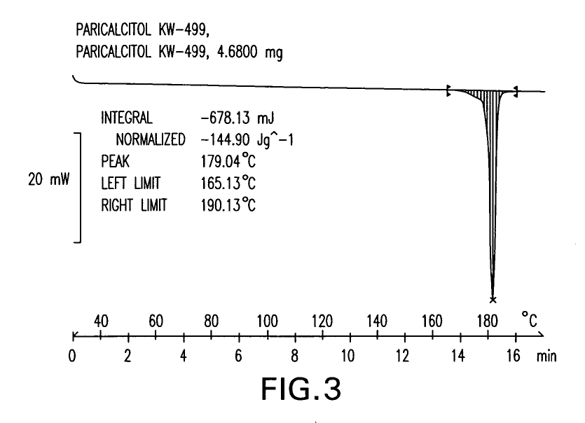

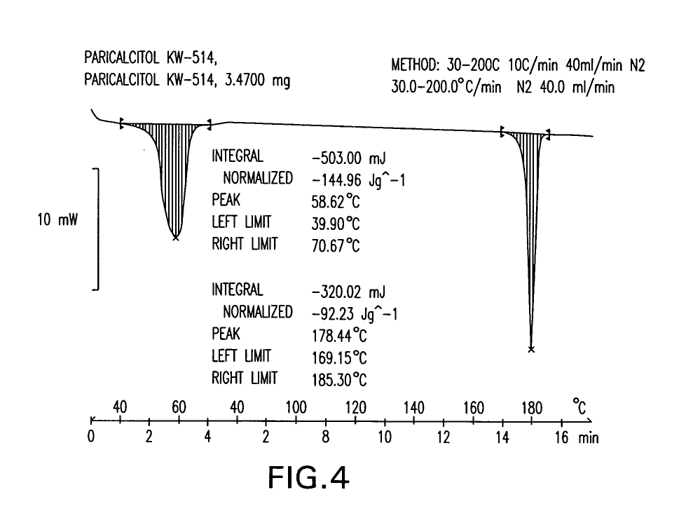

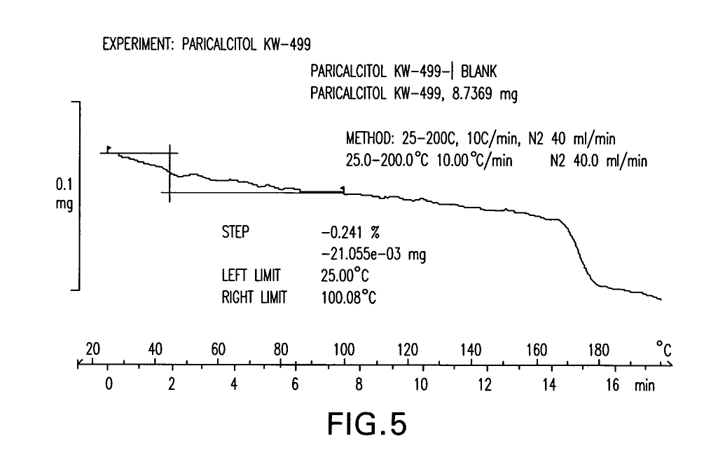

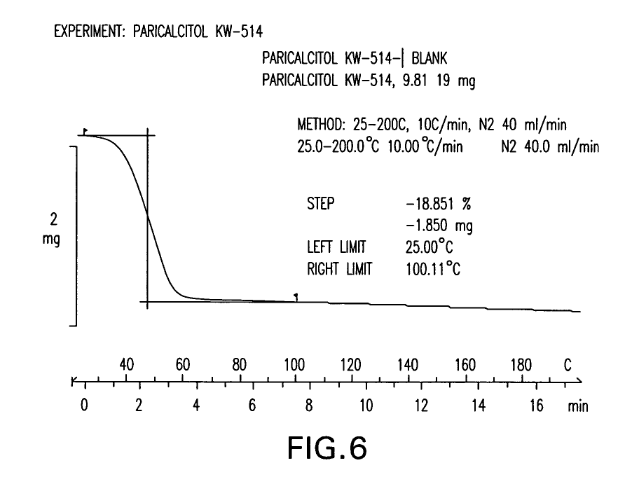

Paricalcitol form II can also be characterized by thermogravimetric analysis (TGA) and differential scanning calorimetry (DSC). FIG. 3 is a representative DSC thermogram of paricalcitol form I. FIG. 4 is a representative DSC thermogram of paricalcitol form II in admixture with form I. The DSC thermogram in FIG. 4, has a low temperature endotherm at about 59° C. and a high temperature endotherm at about 179° C. FIG. 5 is a representative TGA thermogram of paricalcitol form I. FIG. 6 is a representative TGA thermogram of paricalcitol form II in admixture with form I. The low temperature endotherm at about 59° C. in FIG. 4 is believed to be due to loss of solvent of crystallization from the crystal lattice of form II. This conclusion is fully consistent with the results of TGA measurements, which clearly show a distinct step-loss in sample weight at about the temperature of the low-temperature DSC endotherm.

Moreover, in particular embodiments crystalline paricalcitol form II is a tert-butanol (2-methyl-propan-2-ol) solvate. The amount of tert-butanol in the crystal is more than about 1%.

In another aspect, the present invention provides a mixture of crystalline paricalcitol forms I and II, having a powder X-ray diffraction pattern comprises peak reflections at about 5.4°, 8.0°, 11.6°, 14.2°, 15.2°, 17.8°, 18.3°, and 19.0°±0.2° 2θ. Further, the mixture of crystalline paricalcitol forms I and II, can be characterized having additional peak powder X-ray reflections at about 4.70°, 6.9°, 9.5°, 10.8°, 12.6°, and 17.4°±0.2° 2θ. The ratio of crystalline paricalcitol form I to form II in the mixture of crystalline paricalcitol according to the present invention can be from about 10% form I: 90% form II to about 40% form I: 60% form II.

In yet another aspect, the present invention provides a process of preparing a mixture of crystalline paricalcitol forms I and II by crystallization from tert-butanol. In one embodiment, the process comprises dissolving paricalcitol in tert-butanol to form a solution, concentrating the solution, cooling the solution and recovering the mixture of crystalline paricalcitol forms I and II.

In this embodiment, paricalcitol is preferably dissolved in tert-butanol by stirring and heating the solvent. Preferably, heating the solvent at a temperature of about 27° C. to about 45° C., more preferably of about 30° C. to about 35° C. In the heated solvent paricalcitol is preferably dissolved by stirring for about 20 minutes to about 40 minutes, more preferably for a period of 30 minutes.

Preferably, the solution is cooled to a temperature of about 24° C. to about 26° C., more preferably to about 25° C.

Preferably, the cooled solution is stirred for a period of about 30 minutes to about 20 hours, more preferably about 6 hours, even more preferably about 1 hour.

Preferably, the concentrated solution has a concentration of about 0.85% to about 1.4%, more preferably of about 0.9% to about 1.2%, on a weight per volume (i.e. g/mL, g Paricalcitol/mL solvent) basis. Preferably, the step of concentrating the solution is by evaporation. Moreover, concentrating the solution is preferably from a solution of paricalcitol having a concentration of about 0.5% to about 0.6% w/v, more preferably of about 0.6% w/v.

The obtained mixture of crystalline Forms I and II is isolated from a slurry that results from the cooling step by any means known in the art, for example by filtration (gravity or suction) or by centrifugation. Preferably, the precipitated crystals are filtered and dried to obtain crystalline paricalcitol. More preferably, the crystalline paricalcitol is dried for a period of about 4 hours to about 24 hours at a temperature of about 25° C. to about 45° C. under vacuum (pressure of less than about 5 mmHg).

The present invention further provides a method for preparing a pharmaceutical composition comprising mixing Paricalcitol prepared according to method of the present invention, and a pharmaceutically acceptable carrier. As used herein, the term “pharmaceutical composition” includes tablets, pills, powders, liquids, suspensions, solutions, emulsions, granules, capsules, suppositories, or injection preparations.

The pharmaceutical composition may be prepared in any dosage form such as a compressed granulate in the form of a tablet for example. Also, uncompressed granulates and powder mixes that are obtained by the method of the present invention in the pre-compression steps can be simply provided in a dosage form of a capsule or sachet. Therefore, dosage forms of pharmaceutical formulations prepared by the method of the present invention include solid dosage forms like tablets, powders, capsules, sachets, troches and losenges.

Preferably, the pharmaceutical composition is formulated into pharmaceutical formulations such as conventional dosage forms, including tablets and capsules. Tablets are preferred dosage forms. In addition, the tablets may be coated with an optional cosmetic tablet coating. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Preferably, the method of the present invention produces compressed solid dosage forms. There are three well known processes for manufacturing such dosage forms; (i) direct compression, (ii) dry granulation and (iii) wet granulation. There are two well known processes for wet granulation. A wet granulate can be prepared using a mixer and subsequently the wet granulate is dried in order to obtain a dry homogenous granulate. In another method a wet granulate is prepared by spray granulation. In a fluid-bed, spray granulation process, particles and granulate are built up in a fluid bed by spraying a liquid onto fluidized particles. Thus in such process materials are fluidized in the fluid bed dryer and subsequently a solution is sprayed through a nozzle. The choice of processing approach depends upon the properties of the drug and chosen excipients, for example particle size, blending compatibility, density and flowability.

Moreover, the present invention also comprises pharmaceutical compositions comprising the designated forms of crystalline paricalcitol of the present invention, and at least one pharmaceutically acceptable excipient. Preferably, such pharmaceutical composition comprises crystalline paricalcitol form II of the present invention. In an alternative composition the pharmaceutical composition comprises a mixture of crystalline paricalcitol forms I and II. In addition, pharmaceutical compositions of the present invention can contain excipients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quatemary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid compositions and liquid suspension compositions can also be dyed using any pharmaceutically acceptable colorant to improve their appearance and/or facilitate patient identification of the product and unit dosage level.

In liquid suspension pharmaceutical compositions comprising crystalline paricalcitol form II or a mixture of crystalline paricalcitol form I and form II of the present invention, the crystalline paricalcitol of the present invention is suspended, retaining its crystalline form, and any other solid ingredients are either dissolved or suspended in a liquid carrier, such as water, vegetable oil, alcohol, polyethylene glycol, propylene glycol or glycerin. Moreover, a suitable liquid carrier, either with or without other pharmaceutical excipients dissolved therein, for use in the liquid pharmaceutical composition is selected such that the crystalline paricalcitol of the present invention is suspended and not dissolved in such liquid carrier.

Liquid suspension pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid suspension pharmaceutical compositions comprising crystalline paricalcitol form II or a mixture of crystalline paricalcitol form I and form II of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at safe levels to improve storage stability.

A liquid suspension composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

The present invention further encompasses a process for preparing a pharmaceutical formulation comprising combining the designated forms of crystalline paricalcitol of the present invention with at least one pharmaceutically acceptable excipient.

The present invention also encompasses the use of the designated forms of crystalline paricalcitol of the present invention, for the manufacture of a pharmaceutical composition.

In yet another aspect the present invention provides a method of preparing a pharmaceutical composition comprising crystalline paricalcitol form II comprising the steps of
a) providing crystalline paricalcitol comprising crystalline paricalcitol form II; and
b) mixing the crystalline paricalcitol with at least one pharmaceutical acceptable excipient.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as buffer agents, and analgesic agents may be added. If necessary, coloring agents, preservatives, perfumes, seasoning agents, sweetening agents, and other medicines may also be added to the desired preparations.

A pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (suspensions), and the like.

The amount of crystalline paricalcitol of the present invention contained in a pharmaceutical composition according to the present invention is not specifically restricted; however, the dose should be sufficient to treat, ameliorate, or reduce the condition.

The present invention also provides a method of treating a patient suffering from an illness comprising administering to the patient in need thereof a therapeutically effective amount of a pharmaceutical composition comprising crystalline paricalcitol form II and at least one pharmaceutical acceptable excipient. In a preferred method the patient is suffering from an illness selected from secondary hyperthyroidism and plaque psoriasis.

Having described the invention with reference to certain preferred embodiments, other embodiments will become apparent to one skilled in the art from consideration of the specification. The disclosures of the prior art references referred to in this patent application are incorporated herein by reference. The invention is further defined by reference to the following examples describing in detail the preparation of the compound of the present invention. It will be apparent to those skilled in the art that many modifications, both to materials and methods, may be practiced without departing from the scope of the invention.

### EXAMPLES

HPLC Method:

Column: Hypersyl Gold (250×4.6 5 μm)
Mobile phase: (A) water (95%)
   (B) acetonitrile (5%)
Gradient: From 0 to 10 min (A) isocraticaly
   From 10 to 30 min (B) increases from 0 to 55%
   From 30 to 40 min (A) isocraticaly
   From 30 to 40 min (B) increases from 55 to 100%
Detection: 252 nm
Flow: 2 mL/min
Detection limit: 0.02%
   XRD

X-Ray Powder diffraction diagrams were obtained using a Scintag X-Ray powder diffractometer model X'TRA equipped with a Cu-tube and a solid state detector. Samples for analysis were contained in a round standard sample holder with round zero background plate. The scanning parameters were: range: 2-40 deg.2θ: continuous scan, rate: 3 deg./min.

Thermal Analysis

Differential Scanning Calorimetry (DSC) was performed on DSC821^{e}, Mettler Toledo calorimeter. Samples for DSC analysis were contained in a crucible that was crimped closed and punched prior to analysis. Sample weights were in the range of 3-5 mg. The heating rate was 10° C./min.

TGA

Thermogravimetric analysis (TGA) was performed using a Mettler TG50 instrument. Analysis was performed at a heating rate of 10° C./min. The sample weight was 7-15 mg.

The DSC thermogram for Paricalcitol form II exhibits an endothermic peak at about 59° C. This peak probably originates from the loss of solvent, as suggested by a weight loss step in the TGA curve at a similar temperature.

### Example 1

### Crystallization of Paricalcitol from Acetone

500 mg of Paricalcitol were dissolved in 75 ml of acetone in a sonicator at 28° C. over a period of 15 minutes. The clear solution was filtered through glass wool into another flask, and the solution was then concentrated by evaporation, until the volume was 57.5 ml acetone (control by weight). The solution was cooled to −18° C., and the temperature was maintained at −18° C. for 20 hours. The crystals were filtered and washed with 20 ml of cold (−18° C.) acetone, then dried at high vacuum in an oven at 28° C. for 22 hours to obtain a yield of 390 mg (purity of 98.54%).

### Example 2

### Crystallization of Paricalcitol from Acetone+Water

540 mg of Paricalcitol were dissolved in 81 ml of acetone in a sonicator at 28° C. over a period of 15 minutes. The clear solution was filtered through glass wool into another flask, and 8 ml water was added. The solution was then concentrated by evaporation to a volume of 54 ml of acetone (control by weight). The solution was cooled to −18° C., and that temperature was maintained for 16 hours The crystals were filtered and washed with 20 ml of cold (−18° C.) acetone, and then dried at high vacuum in an oven at 28° C. for 6 hours to obtain a yield of 300 mg (purity of 99.79%).

### Example 3

### Crystallization of Paricalcitol from Ethyl Acetate

520 mg of Paricalcitol were dissolved in 100 ml of Ethyl acetate in a sonicator at 28° C. over a period of 15 minutes. The clear solution was filtered through glass wool into another flask, and the solution was then concentrated by evaporation to a volume of 86 ml of Ethyl acetate (control by weight). The solution was cooled to −18° C., and that temperature was maintained for 20 hours. The crystals were filtered and washed with 20 ml of cold (−18° C.) Ethyl acetate, then dried at high vacuum in an oven at 28° C. for 20 hours to obtain a yield of 360 mg (purity of 98.46%).

### Example 4

### Crystallization of Paricalcitol from Ether-Acetone

1.25 g of Paricalcitol were dissolved in 290 ml of diethyl ether-acetone solution (1:2) with stirring at 34° C. over a period of 30 minutes. The solution was then concentrated by evaporation to a total weight of about 150 g. The solution was cooled to −18° C., and that temperature was maintained for 4 hours. The crystals were filtered and washed with 20 ml of cold acetone (−18° C.), then dried at high vacuum in an oven at 30° C. for 1 hour to obtain a yield of 920 mg.

### Example 5

### Crystallization of Paricalcitol from Ether-Methyl formate-CH₃CN-EtOH

1.07 g of Paricalcitol were dissolved in a mixture of 150 ml Ether, 150 ml Methyl formate, 100 ml CH₃CN, and 20 ml EtOH. The solution was cooled to 0° C., and seeded with crystals of Paricalcitol, cooled to −45° C., and stirred at −45° C. for 1 hour. The crystals were filtered, and then dried at high vacuum in an oven at 28° C. for 2 hours to obtain a yield of 630 mg (purity of 99.38%).

### Example 6

### Crystallization of Paricalcitol from tert-Butanol

100 mg of Paricalcitol were dissolved in 17 ml of tert-Butanol with stirring at 30° C. over a period of 30 minutes. The solution was then concentrated by evaporation at 30° C. to a volume of about 11 ml tert-Butanol (control by weight). The solution was cooled to 25° C., and stirred at that temperature for 1 hour. The crystals were filtered and then dried at high vacuum in an oven at 28° C. for 6-20 hours to obtain a yield of 60 mg (purity of 99.63%) of paricalcitol as a mixture of form I and form II.

### Example 7

### Crystallization of Paricalcitol from tert-Butanol

Six hundred milligrams of paricalcitol were dissolved in 100 mL t-Butanol, at 34° C. over 30 min. The resulting solution was filtered through glass wool to another flask and the solution was then concentrated by evaporation at 34° C. to a volume of 50 ml tert-Butanol (=83 ml per 1 g material—1.2% w/v). The solution was then cooled to 26° C. and stirred at 26° C. for 4 hours. The obtained crystalline material was filtered and dried at 30° C. under vacuum (P˜2 mmHg) for 20 hours, to give 80 mg paricalcitol as a mixture of form I and form II.

### Example 8

### Crystallization of Paricalcitol from Acetone

1.35 g Paricalcitol were dissolved in 270 ml Acetone, at 32° C., with stirring, during 15 min. Then, the solution was filtered through glass wool to another flask and the solvent was carefully evaporated, under reduced pressure at 32° C., until a volume of 218 ml acetone.

Then, the solution was cooled to 10° C. and the solution was seeded with 18 mg Paricalcitol then cooled to −18° C. and stirred at −18° C., at 200 rpm for 16 hours. The obtained crystalline material was filtered, washed with 20 ml cold (−18° C.) acetone, and dried at 28° C. under vacuum (P˜2 mmHg) for 6 hours, to give 900 mg cryst. Paricalcitol.

### Example 9

### Crystallization of Paricalcitol from Acetone

2.35 g Paricalcitol were dissolved in 353 ml Acetone, at 28° C., in the sonicator, during 15 min. Then, the solution was filtered through glass wool to another flask which was put, in the Lauda at 22° C.

Then, stirring was started and the flask was cooled to −18° C. during 12 hours and continue stirring at −18° C., for another 6 hours.

The obtained crystalline material was filtered, washed with 20 ml cold (−18° C.) acetone, and dried at 28° C. under vacuum (P˜2 mmHg) for 6 hours, to give 1.81 g cryst. Paricalcitol.

### Example 10

### Crystallization of Paricalcitol from Ethyl Acetate+Water

0.40 g Paricalcitol was dissolved in 80 ml ethyl acetate, in the sonicator, at 28° C., during 10 min. Then, the solution was filtered through glass wool to another flask, and 6.5 ml water was added. The solvent was carefully evaporated, under reduced pressure at 32° C., until a volume of 66 ml ethyl acetate (=165 volumes, control by weight). Then, the flask was put at −18° C. for 16 hours.

The obtained crystalline material was filtered, washed with 30 ml cold (−18° C.) ethyl acetate, and dried at 28° C. under vacuum (P˜2 mmHg) for 22 hours, to give 0.23 g cryst. Paricalcitol. (purity of 98.88%)

### Example 11

### Crystallization of Paricalcitol from CH₃CN (Acetonitrile)

1.01 g Paricalcitol were dissolved in 200 ml CH₃CN, at 30° C., in the sonicator, during 30 min. Then, the solution was filtered through glass wool to another flask which was put, in the Lauda at 22° C.

Then, stirring was started and the flask was cooled to −18° C. and continue stirring at −18° C., for 18 hours.

The obtained crystalline material was filtered, washed with 20 ml cold (−18° C.) CH₃CN, and dried under vacuum (P˜2 mmHg) at 28° C. for 20 hours, to give 0.6 g cryst. Paricalcitol.

### Example 12

### Crystallization of Paricalcitol from CH₃CN+Water

0.4 g Paricalcitol were dissolved in 160 ml solution of 5% water in CH₃CN, at 30° C., in the sonicator, during 15 min. Then, the solution was filtered through glass wool to another flask which was put, in the Lauda at 22° C.

Then, stirring was started and the flask was cooled to −18° C. and continue stirring at −18° C., for 18 hours.

The obtained crystalline material was filtered, washed with 20 ml cold (−18° C.) CH₃CN, and dried under vacuum (P˜2 mmHg) at 28° C. for 20 hours, to give 0.28 g cryst. Paricalcitol.

1. A method for purifying Paricalcitol comprising the steps of
a) dissolving Paricalcitol in a solvent;
b) cooling the solution to form a precipitate; and
c) recovering precipitate.

2. The method according to claim 1, wherein the solvent is selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof.

3. The method according to claim 2, wherein the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof.

4. The method according to claim 1, wherein Paricalcitol and the solvent in step a) are in a ratio of about 1:150 to about 1:450 g Paricalcitol/ml solvent.

5. The method according to claim 4, wherein the ratio is about 1:150 to about 1:200.

6. The method according to claim 1, wherein dissolving Paricalcitol in a solvent is carried out at a temperature of about 25° C. to about 40° C.

7. The method according to claim 6, wherein the temperature is about 28° C. to about 34° C.

8. The method according to claim 1, further comprising the step of filtering the solution obtained in step a) after dissolving Paricalcitol in a solvent.

9. The method according to claim 1, wherein the solution is cooled to a temperature of about −45° C. to about −10° C.

10. The method according to claim 9, wherein the solution is cooled to a temperature of about −20° C. to about −15° C.

11. The method according to claim 10, wherein the solution is cooled to a temperature of about −18° C.

12. The method according to claim 3, wherein when the solvent is tert-butanol the solution is cooled to a temperature of about 25° C. to about 27° C.

13. The method according to claim 1, wherein the solution is cooled at a rate of not more than about 8° C. per hour.

14. The method according to claim 1, wherein the solution is cooled for a period of about 1 to about 24 hours.

15. The method according to claim 14, wherein the period is about 15 to about 24 hours.

16. The method according to claim 15, wherein the period is about 16 to about 20 hours.

17. The method according to claim 1, wherein dissolving Paricalcitol in a solvent is carried out in a sonicator.

18. The method according to claim 1, the method further comprises concentrating the solution from step a) before cooling the solution.

19. The method according to claim 18, wherein the solution is concentrated to about 0.5 to about 0.9 times its original volume.

20. The method according to claim 19, wherein the solution is concentrated to about 0.6 to about 0.8 times its original volume.

21. The method according to claim 19, wherein Paricalcitol and the solvent are in a ratio of about 1:100 to about 1:120 g Paricalcitol: ml solvent.

22. The method according to claim 1, wherein when the method further comprises seeding the solution with crystals of Paricalcitol either before or during the step of cooling the solution.

23. Paricalcitol prepared according to the method of claim 1, wherein the Paricalcitol has a purity of at least about 98% by weight.

24. The Paricalcitol according to claim 23, wherein the Paricalcitol has a purity of at least about 99% by weight.

25. A method of preparing a pharmaceutical composition of Paricalcitol comprising mixing Paricalcitol prepared according to claim 1 with a pharmaceutically acceptable carrier.

26. A crystalline form of paricalcitol (form II) characterized by a powder X-ray diffraction pattern having peak reflections at about 4.7°, 8.0°, 11.6°, 17.4°, 18.3°, and 19.0°±0.2° 2θ.

27. The crystalline form of paricalcitol of claim 26, wherein the crystalline form is characterized by peaks at about 6.9°, 9.5°, and 12.6°±0.2° 2θ.

28. The crystalline form of paricalcitol of claim 26, having a DSC thermogram with an endotherm at about 59° C.

29. The crystalline form of paricalcitol of claim 26, wherein the crystalline paricalcitol is a tert-butanol solvate.

30. The crystalline form of paricalcitol of claim 29, wherein the crystalline form contains more than 1% t-butanol by weight.

31. A mixture of crystalline forms of paricalcitol, wherein the mixture is characterized by a powder X-ray diffraction pattern having peak reflections at about 5.4°, 8.0°, 11.6°, 14.2°, 15.2°, 17.8°, 18.3°, and 19.0°±0.2° 2θ.

32. The mixture of crystalline paricalcitol of claim 31, wherein the mixture is further characterized by peak reflections at about 4.7°, 6.9°, 9.5°, 10.8°, 12.6°, and 17.4°±0.2° 2θ.

33. The mixture of crystalline paricalcitol of claim 32, wherein the mixture has a powder X-ray diffraction pattern as substantially depicted in FIG. 2.

34. The mixture of crystalline paricalcitol of claim 31, having a DSC thermogram with a endotherms at about 59° C. and about 179° C.

35. The mixture of crystalline paricalcitol of claim 31, wherein the mixture has a TGA thermogram as substantially depicted in FIG. 6.

36. A process of preparing the mixture of claim 31, comprising crystallizing paricalcitol from tert-butanol.

37. The process of claim 36, wherein the process comprising the steps of
a) dissolving paricalcitol in tert-butanol to form a solution;
b) concentrating the solution;
c) cooling the solution; and
d) recovering the mixture of crystalline paricalcitol forms I and II.

38. The process of claim 37, wherein the paricalcitol is dissolved in tert-butanol at a temperature of about 27° C. to about 45° C.

39. The process of claim 38, wherein the paricalcitol is dissolved in tert-butanol at a temperature of about 30° C. to about 35° C.

40. The process of claim 37, wherein the solution is cooled to a temperature of about 24° C. to about 26° C.

41. The process of claim 40, wherein the solution is cooled to a temperature of about 25° C.

42. The process of claim 37, wherein the cooled solution is stirred for about 0.5 hours to about 6 hours.

43. The process of claim 37, wherein the cooled solution is stirred for about 1 hour.

44. The process of claim 37, wherein concentrating the solution is to obtain a concentrated solution with a concentration of about 0.85% to about 1.4% w/v of paricalcitol.

45. The process of claim 44, wherein concentrating the solution is to obtain a concentrated solution with a concentration of about 0.9% to about 1.2% w/v of paricalcitol.

46. The process of claim 37, wherein the solution is concentrated by evaporating the tert-butanol.

47. The process of claim 37, wherein before concentration, the solution is that of about 0.5% to about 0.6% w/v of paricalcitol.

48. The process of claim 37, wherein the steps of concentrating the solution and cooling the solution are in a sequential order of first concentrating the solution followed by cooling the solution.

49. The process of claim 37, wherein recovering comprises filtering a slurry, and drying the obtained filtrate at a temperature of about 25° C. to about 45° C. under vacuum for about 4 hours to about 24 hours.

50. A pharmaceutical composition comprising crystalline paricalcitol form II and at least one pharmaceutical acceptable excipient.

51. A method of preparing the pharmaceutical composition of claim 50 comprising admixing crystalline paricalcitol form II with at least one pharmaceutical acceptable excipient.

52. The pharmaceutical composition of claim 50, wherein the crystalline paricalcitol form II is in a mixture of crystalline paricalcitol forms I and II.

53. A method of treating secondary hyperthyroidism and plaque psoriasisa in a mammal in need thereof comprising administering to the patient the pharmaceutical composition of claim 50.
